(19) United States
(12) Patent Application Publication
(10) Pub. No.: US 2014/0025131 A1
(43) Pub. Date: Jan. 23, 2014

(54) WEARABLE DEFIBRILLATOR WITH VOICE PROMPTS AND VOICE RECOGNITION

(71) Applicant: Physio-Control, Inc., Redmond, WA (US)

(72) Inventors: Joseph L. Sullivan, Kirkland, WA (US); Martin S. Abbenhouse, Kirkland, WA (US)

(73) Assignee: PHYSIO-CONTROL, INC., Redmond, WA (US)

(21) Appl. No.: 13/947,747

(22) Filed: Jul. 22, 2013

Related U.S. Application Data
(60) Provisional application No. 61/674,150, filed on Jul. 20, 2012.

(51) Int. Cl. A61N 1/39

(52) U.S. Cl. CPC A61N 1/3993
USPC 607/5

(57) ABSTRACT

An external medical device such as a wearable defibrillator can include a housing, an energy storage module within the housing for storing an electrical charge, and a defibrillation port within the housing for guiding via electrodes the stored electrical charge to a person. The medical device can also include a processor within the housing configured to control a voice prompt customization module, in which the voice prompt customization module allows a programmer to customize voice prompts, and a user interface configured to deliver at least one voice prompt customized by the programmer

### RELATIONSHIP WITH OTHER APPLICATIONS

The present patent application claims the benefit of U.S. Provisional Patent Application Ser. No. 61/674,150, filed on Jul. 20, 2012, the disclosure of which is hereby incorporated by reference for all purposes.

### FIELD

The present subject matter generally relates to the field of medical devices such as wearable defibrillators.

### BACKGROUND

In humans, the heart beats to sustain life. In normal operation, the heart propels blood through the various parts of the body. The chambers of the heart contract and expand in a periodic, regular, and coordinated fashion. The sequence is as follows; The right atrium sends deoxygenated blood into the right ventricle. The right ventricle pumps the blood to the lungs, where, before returning to the left atrium, the blood becomes oxygenated. The left atrium pumps the oxygenated blood to the left ventricle. The left ventricle then expels and forces the blood to circulate through the various parts of the body.

The heart chambers pump because of the heart's electrical control system. More particularly, the sinoatrial (SA) node generates an electrical impulse, which cascades into electrical signals. The electrical signals in turn cause the above-described contractions of the various chambers in the heart in the correct sequence. The electrical pattern created by the sinoatrial (SA) node is called a sinus rhythm.

Unfortunately, sometimes the electrical control system of the heart malfunctions and causes the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the rest of the body. Some arrhythmias may even result in a Sudden Cardiac Arrest (SCA). In a SCA, the heart fails to pump blood effectively and, if not treated, death can occur. It is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, chaotic, spasm-like movements, instead of the normal, coordinated, sequential rhythmic contractions. When arrhythmia happens, the heart does not pump enough blood to deliver enough oxygen to the vital organs. The person's condition deteriorates rapidly and, if not reversed, the person may expire within minutes.

Ventricular Fibrillation can often be reversed using a life-saving device called a defibrillator. A defibrillator, when applied properly and promptly, can administer an electrical shock to the heart and terminate the VF, giving the heart an opportunity to resume proper functioning. If the VF is not terminated with the initial shock, subsequent shock may be administered, often at escalating energies.

A challenge with defibrillation is that the electrical shock, if not immediately at the onset, must be administered as soon as possible right after the onset of the VF. There is not much time. The survival rate of persons suffering from VF decreases by about 10% for each minute the administration of a defibrillation shock is delayed. After about 10 minutes, the rate of survival for SCA victims averages less than 2%.

During VF, a person's condition deteriorates rapidly because the blood is not flowing to the brain, heart, lungs, and other organs. If resuscitation attempts are to be successful and damage to organs prevented, blood flow must be restored.

To-date, the challenge of administering a shock and defibrillating as quickly as possible and within minutes of the onset of VF has been approached in a number of ways. Great efforts and training are constantly being implemented to ensure as short of an emergency teams response time as possible. Great efforts and education are being implemented in communities to empower lay bystanders to respond to such events as quickly and efficiently as possible.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. CPR can be beneficial for persons experiencing VF because it slows the deterioration that would otherwise occur while a defibrillator is being retrieved. Indeed, for patients with an extended down-time, survival rates are higher if CPR is administered prior to defibrillation. Advanced medical devices often can also coach a rescuer who performs CPR. For example, a medical device can issue instructions, and even prompts, for the rescuer to perform CPR more effectively. VF can occur unpredictably, even to a person who is not considered at a high risk and cardiac events can be experienced by people who lack the benefit of an Implantable Cardioverter Defibrillator (ICD) therapy.

For people who are considered to be at a higher risk of VF or other heart arrhythmias, when indicated, an ICD can be implanted surgically. An ICD can monitor the person's heart, and administer an electrical shock promptly. An ICD reduces the need to have the higher-risk person be monitored constantly by medical personnel.

When VF occurs to a person who does not have an ICD, they collapse, because blood flow has stopped. They should receive therapy quickly. For a VF victim without an ICD, a different type of defibrillator can be used, which is called an external defibrillator. External defibrillators have been made portable, so they can be brought by a bystander/rescuer to a potential VF victim quickly enough to revive them. The patient's life may hinge on the bystander/rescuer quick and efficient response to the situation. The time from the collapse to the time a portable defibrillator is applied to the cardiac event victim is critical.

### BRIEF SUMMARY

In certain embodiments, an external medical device such as a wearable defibrillator may include a housing, an energy storage module within the housing for storing an electrical charge, and a defibrillation port for guiding via electrodes the stored electrical charge to a patient. The device may also include a user interface to deliver voice prompts, e.g., to the patient, a family member of the patient, or a rescuer. A voice prompt customization module of the defibrillator may provide a programmer, e.g., a medical professional, with the ability to customize various aspects of the voice prompts such as tone, volume, language, and style, as well as characteristics of the spoken voice such as gender and ethnicity. A voice prompt database of the defibrillator may store the customized voice prompts.

An advantage over the prior art is that voice prompts from a medical device, such as a wearable defibrillator, may be tailored specifically to the individual to whom the device is prescribed. Simply referring to a certain patient by name will generally do a better job of getting the patient's attention than would be possible with a generic, non-customized voice prompt. In certain embodiments, specific voice prompts or types of voice prompts may be tailored to be commanding, reassuring, etc. based on the personality of the patient. Alternatively or in addition thereto, the device may distinguish a response spoken by the patient from words spoken by other people.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing different types of the external defibrillator shown in FIG. 1, and who they might be used by.

FIG. 3 is a functional block diagram showing components of an external defibrillator, such as the one shown in FIG. 1.

FIG. 4 is a block diagram showing a patient interacting with a user interface of an external defibrillator according to embodiments.

FIG. 5 is a block diagram showing a patient receiving a customized voice prompt from an external defibrillator according to embodiments.

FIG. 6 is a block diagram showing a patient receiving another customized voice prompt from an external defibrillator according to embodiments.

FIG. 7A is a block diagram showing a patient receiving from an external defibrillator a voice prompt requesting a response by the patient according to embodiments.

FIG. 7B is a block diagram showing the patient responding to the voice prompt issued by the external defibrillator in FIG. 7A.

FIG. 8 is a flowchart for illustrating example methods executable by external medical devices according to embodiments.

FIG. 9 is a flowchart for illustrating example methods executable by external medical devices according to embodiments.

### DETAILED DESCRIPTION

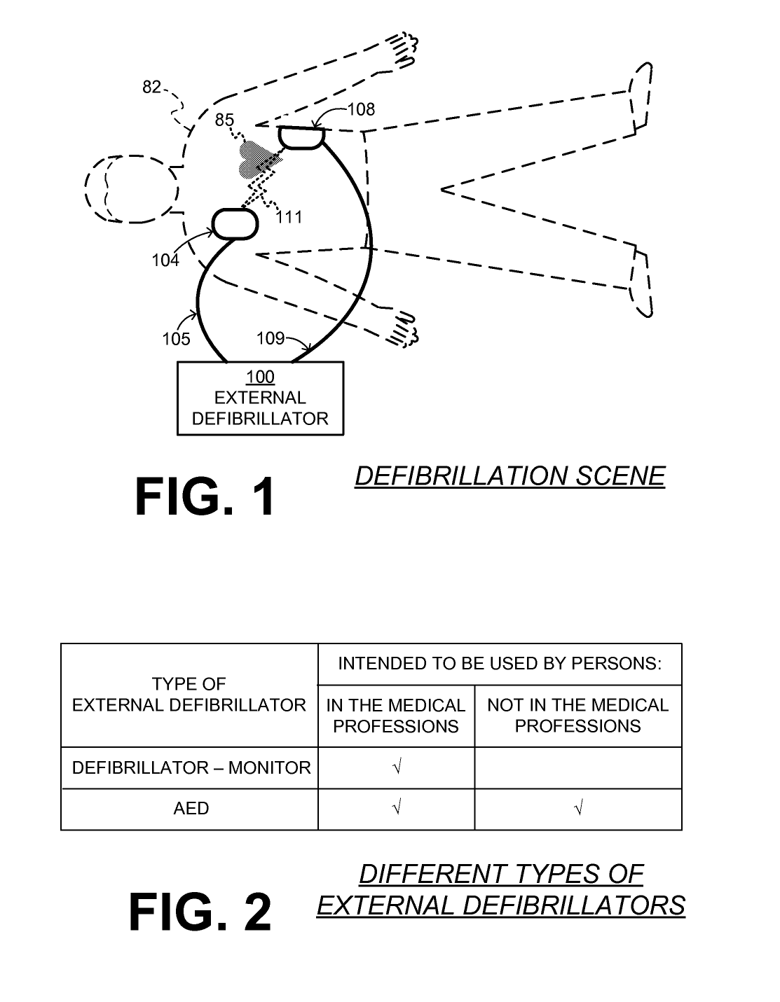

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing different types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. For example, one such aspect is additional ECG monitoring capability.

For patients who qualify for the invasive surgical procedure and an ICD, there is a wait time from the point of diagnosis to the point of the surgical placement of an ICD in a patient. If not monitored, the wait period renders the patient vulnerable to life threatening cardiac episodes. For patients who are vulnerable to cardiac episodes yet are not good candidates for surgery, however, another type of solution, such as by way of an example a wearable defibrillator/monitor, would be highly desirable.

Thus, there is a pressing need for a system, device, method for an automated, continual, and relative to an ICD, non-invasive monitoring and, upon need, immediate therapy administration to a cardiac event victim. As such, a pressing need exists for an improved approach to collecting, storing, transferring to a remote location/medical professional, and analyzing data to ensure quick and accurate medical response to an emergency situation.

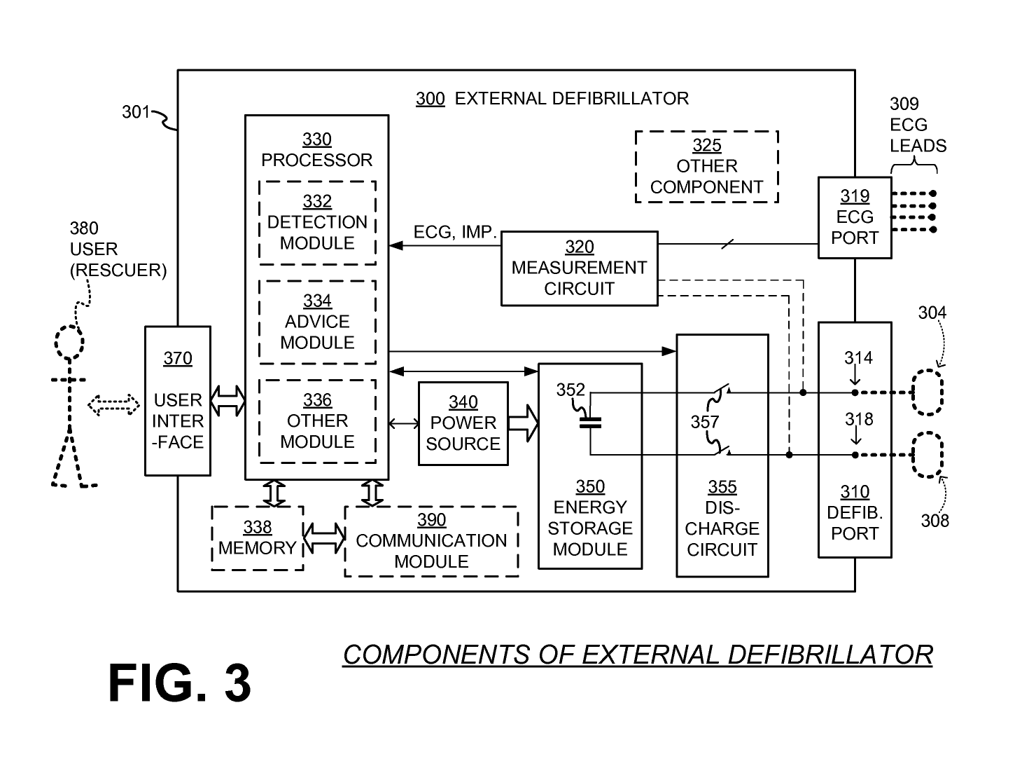

FIG. 3 is a diagram showing components of an external defibrillator 300. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer, or the person 82. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be described later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and an other component 325 for the above described additional features, such as patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. In certain embodiments, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, device information, incident information, therapy attempted, CPR performance, and so on.

Defibrillators and, in particular, wearable defibrillators occasionally rely on the use of voice prompts to help a patient avoid receiving inappropriate shocks. When a defibrillator detects a shockable rhythm in a patient, for example, the defibrillator may alert the patient and instruct the patient to perform a certain action, such as pressing a button, to avoid being shocked. These patients are often not confident about technology and tend to be easily confused. Enabling a patient to custom-tailor voice prompts to be issued to the patient by the defibrillator can ensure that the patient understands and complies with voice prompts issued by the defibrillator.

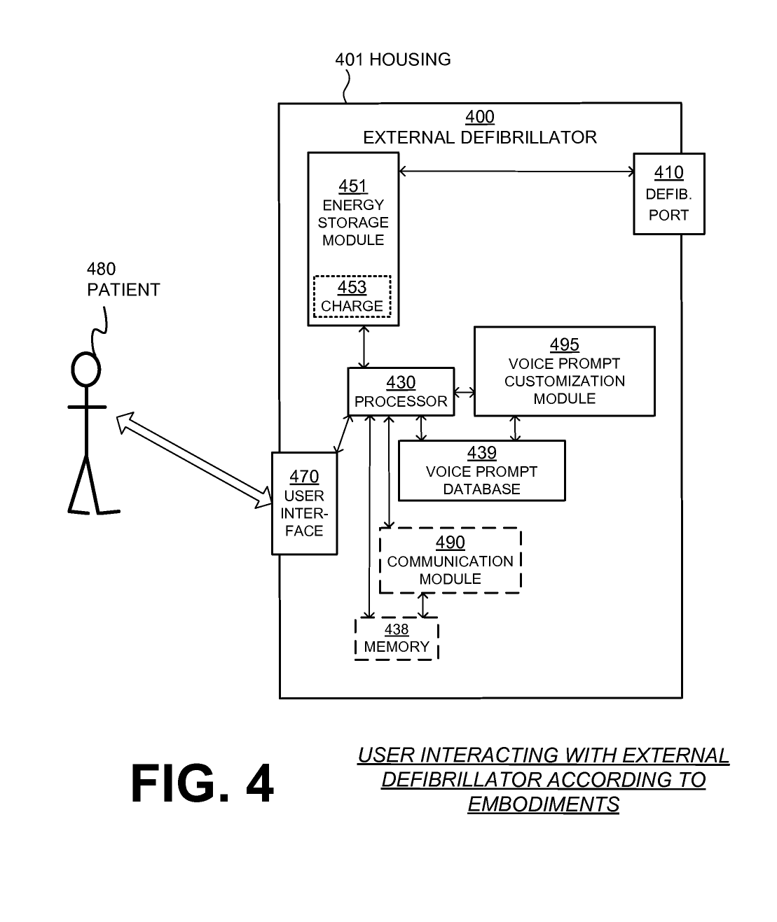

FIG. 4 is a block diagram showing a patient 480 interacting with a user interface 470 of an external defibrillator 400 according to embodiments. In certain embodiments, the external defibrillator 400 may be configured to be worn by the patient 480.

The external defibrillator 400 includes a housing 401, a processor 430, an energy storage module 451 in an interior of the housing 401 for storing an electrical charge 453, and a defibrillation port 410 for guiding via electrodes the electrical charge 453 to the patient 480.

In certain embodiments, the external defibrillator 400 may include a memory 438 for storing medical data or other information. Alternatively or in addition thereto, the defibrillator 400 may include a communication module 490 for facilitating communication between the defibrillator 400 and one or more other devices or location, such as an emergency center.

In the example, the external defibrillator 400 includes a voice prompt database 439 for storing voice prompts to be delivered to the patient 480 by way of the user interface 470. A voice prompt customization module 495 allows a programmer to customize voice prompts, such as those stored by the voice prompt database 439, to be delivered by the defibrillator 400.

As used herein, the term programmer generally refers to a user, such as a medical professional, that is authorized to interact with the voice prompt customization module 495. In certain embodiments, the patient 480 may be the programmer, e.g., in situations where previously customized voice prompts are further customized or otherwise altered.

In certain embodiments, the wording of voice prompts to be delivered by the defibrillator can be customized to suit the needs and/or preferences of the patient 480 and also to improve compliance by the patient 480 therewith. For example, the programmer can specify that a certain voice prompt or certain type of voice prompts be more verbose (e.g., “Please press the big red button—a shock will be delivered if the button is NOT pressed”) or more terse (e.g., “Press the big red button now!”). Default levels of verbosity and terseness may be established to provide a reference point for the programmer in specifying the desired levels thereof for a given voice prompt or type of voice prompt.

In certain embodiments, the tone quality of voice prompts to be delivered by the defibrillator 400 can be altered, e.g., to compensate for selective hearing loss. Such altering can be performed automatically by the defibrillator 400 or pursuant to a particular instruction or request from the patient 480. Alternatively or in addition thereto, the gender and/or language in which voice prompts are delivered to the patient 480 can be tailored to suit certain requirements and/or preferences of the patient 480. For example, if the patient 480 speaks only in Spanish, he or she can specify that all voice prompts, regardless of what type of voice prompt, be delivered in Spanish.

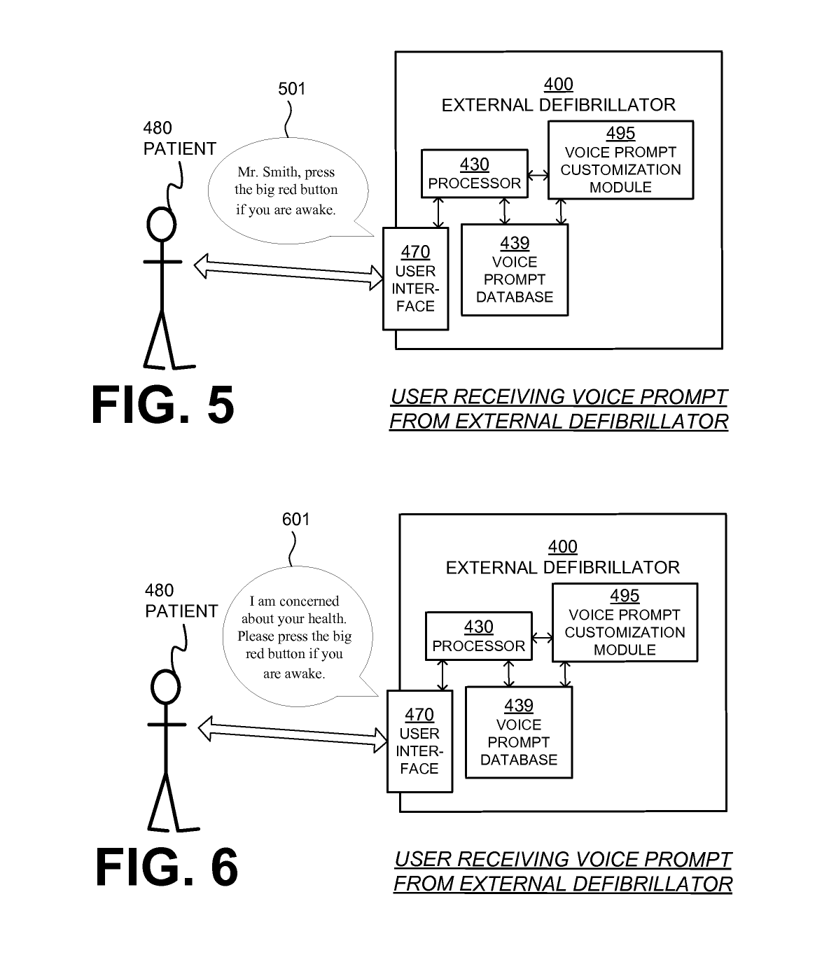

In certain embodiments, an external defibrillator such as a wearable defibrillator can be customized to refer to the patient by name. FIG. 5 is a block diagram showing the patient 480 of FIG. 4 receiving such a voice prompt 501 from the external defibrillator 400 according to embodiments. In the example, the processor 430 and voice prompt customization module 495 of the defibrillator 400, in connection with the voice prompt database 439, cause the user interface 470 to issue the voice prompt 501 that refers to the patient 480 by name.

In other embodiments, such as when an action needs to be performed on the patient rather than by the patient, the voice prompt(s) may be customized to refer to one or more other people, such as a spouse or children of the patient, by name. An example of such a voice prompt for patient Mr. Smith is “Mrs. Smith, you need to do chest compressions on your husband now!”

In certain embodiments, an external defibrillator such as a wearable defibrillator can be customized to refer to itself in the first person. FIG. 6 is a block diagram showing the patient 480 of FIG. 4 receiving such a voice prompt 601 from the external defibrillator 400 according to embodiments. In the example, the processor 430 and voice prompt customization module 495 of the defibrillator 400, in connection with the voice prompt database 439, cause the user interface 470 to issue a voice prompt 601 that gives the appearance of the defibrillator 400 referring to itself in the first person.

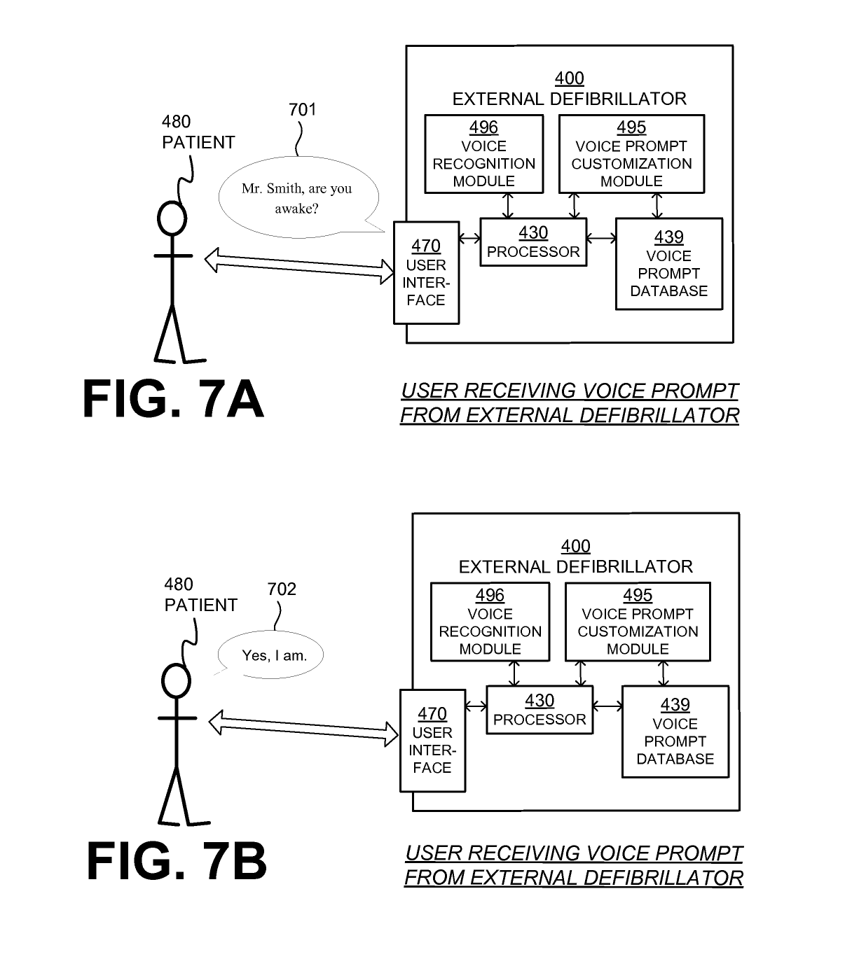

In certain embodiments, an external defibrillator such as a wearable defibrillator can be configured to recognize the patient's verbal response to a voice prompt issued by the defibrillator. FIG. 7A is a block diagram showing the patient 480 of FIG. 4 receiving from the external defibrillator 400 a voice prompt 701 that requests a response thereto by the patient 480 according to embodiments. In the example, the processor 430 and voice prompt customization module 495 of the defibrillator 400, in connection with the voice prompt database 439, cause the user interface 470 to issue the voice prompt 701 that requests a response by the patient.

FIG. 7B is a block diagram showing the patient responding 702 to the voice prompt 701 issued by the external defibrillator 400 in FIG. 7A. In certain embodiments, a voice recognition module 496 of the defibrillator 400 can be trained to specifically recognize the voice of the patient 480. Alternatively or in addition thereto, the voice recognition module 496 can be trained to recognize the voice of one or more other people, such as a child or spouse of the patient 480. In such embodiments, the external defibrillator 400 may automatically determine which voice prompt(s) are to be delivered by the defibrillator—as well as which characteristics thereof, such as language, gender, tone quality, etc. based on who the defibrillator identifies.

In certain embodiments, the voice recognition module 496 may be configured such that, in connection with issuing a voice prompt, it may also actively listen for certain questions and/or phrases, such as any or all of the following (or variations thereof): “What?”, “I don't understand!”, “I don't know what to do!”, and “Please repeat.” In response to identifying such questions/phrases, the voice recognition module 496 may cause the defibrillator 400 to perform a corresponding action, e.g., issue another prompt or repeat the previously issued prompt. The voice recognition module 496 may be configured to only monitor for questions/phrases spoken by the patient, people other than the patient, or both. In certain embodiments, the voice recognition module 496 may be further configured to identify the language in which such a question/phrase is spoken and then perform the corresponding action, e.g., repeat the prompt, using the same language as that used by the speaker of the question/phrase.

In certain embodiments, the external defibrillator may have a name or other designation assigned to it so that the defibrillator may be addressed by the name, for example. In such embodiments, the name may be selected or created by the patient or someone else, or it may be predetermined or automatically generated.

In certain embodiments, the programmer may configure the defibrillator to provide a voice prompt in two or more languages. If the patient's family has some English-only speakers and some Spanish-only speakers, for example, the defibrillator can be configured to provide each voice prompt twice—once in English and once in Spanish—when delivered by the defibrillator.

The functions of this description may be implemented by one or more devices that include logic circuitry. The device performs functions and/or methods as are described in this document. The logic circuitry may include a processor that may be programmable for a general purpose, or dedicated, such as microcontroller, a microprocessor, a Digital Signal Processor (DSP), etc. For example, the device may be a digital computer like device, such as a general-purpose computer selectively activated or reconfigured by a computer program stored in the computer. Alternately, the device may be implemented by an Application Specific Integrated Circuit (ASIC), etc.

Moreover, methods are described below. The methods and algorithms presented herein are not necessarily inherently associated with any particular computer or other apparatus. Rather, various general-purpose machines may be used with programs in accordance with the teachings herein, or it may prove more convenient to construct more specialized apparatus to perform the required method steps. The required structure for a variety of these machines will become apparent from this description.

In all cases there should be borne in mind the distinction between methods in this description, and the method of operating a computing machine. This description relates both to methods in general, and also to steps for operating a computer and for processing electrical or other physical signals to generate other desired physical signals.

Programs are additionally included in this description, as are methods of operation of the programs. A program is generally defined as a group of steps leading to a desired result, due to their nature and their sequence. A program is usually advantageously implemented as a program for a computing machine, such as a general-purpose computer, a special purpose computer, a microprocessor, etc.

Storage media are additionally included in this description. Such media, individually or in combination with others, have stored thereon instructions of a program made according to certain embodiments. A storage medium according to certain embodiments is a computer-readable medium, such as a memory, and is read by the computing machine mentioned above.

Performing the steps or instructions of a program requires physical manipulations of physical quantities. Usually, though not necessarily, these quantities may be transferred, combined, compared, and otherwise manipulated or processed according to the instructions, and they may also be stored in a computer-readable medium. These quantities include, for example electrical, magnetic, and electromagnetic signals, and also states of matter that can be queried by such signals. It is convenient at times, principally for reasons of common usage, to refer to these quantities as bits, data bits, samples, values, symbols, characters, images, terms, numbers, or the like. It should be borne in mind, however, that all of these and similar terms are associated with the appropriate physical quantities, and that these terms are merely convenient labels applied to these physical quantities, individually or in groups.

This detailed description is presented largely in terms of flowcharts, display images, algorithms, and symbolic representations of operations of data bits within at least one computer readable medium, such as a memory. Indeed, such descriptions and representations are the type of convenient labels used by those skilled in programming and/or the data processing arts to effectively convey the substance of their work to others skilled in the art. A person skilled in the art of programming may use these descriptions to readily generate specific instructions for implementing a program according to certain embodiments of the disclosed technology.

Often, for the sake of convenience, it is preferred to implement and describe a program as various interconnected distinct software modules or features, individually and collectively also known as software.

This is not necessary, however, and there may be cases where modules are equivalently aggregated into a single program with unclear boundaries. In any event, the software modules or features of this description may be implemented by themselves, or in combination with others. Even though it is said that the program may be stored in a computer-readable medium, in view of the present disclosure, it should be clear to a person skilled in the art that it need not be a single memory, or even a single machine Various portions, modules or features of it may reside in separate memories, or even separate machines. The separate machines may be connected directly, or through a network, such as a local access network (LAN), or a global network, such as the Internet.

In view of the present disclosure, it will be appreciated that some of these methods may include software steps that may be performed by different modules of an overall software architecture. For example, data forwarding in a router may be performed in a data plane, which consults a local routing table. Collection of performance data may also be performed in a data plane. The performance data may be processed in a control plane, which accordingly may update the local routing table, in addition to neighboring ones. In view of the present disclosure, a person skilled in the art will discern which step is best performed in which plane.

An economy is achieved in the present document in that a single set of flowcharts is used to describe both programs, and also methods. So, while flowcharts are described in terms of boxes, they can mean both method and programs.

For this description, the methods may be implemented by machine operations. In other words, embodiments of programs are made such that they perform methods that are described in this document.

These may be optionally performed in conjunction with one or more human operators performing some, but not all of them. As per the above, the users need not be collocated with each other, but each only with a machine that houses a portion of the program. Alternately, some of these machines may operate automatically, without users and/or independently from each other.

Methods are now described.

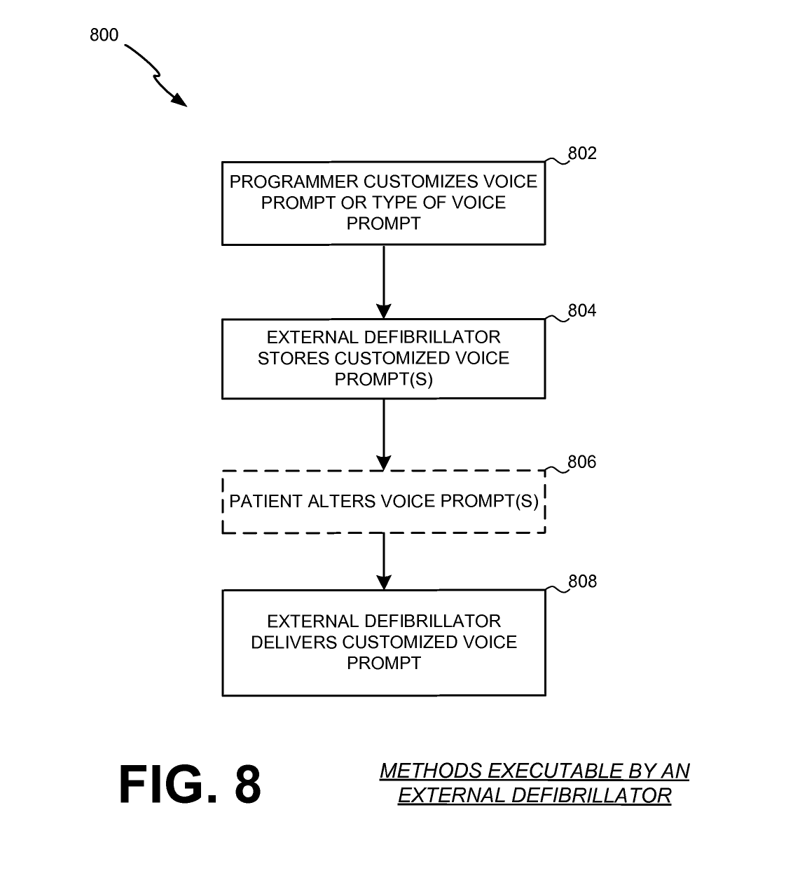

FIG. 8 is a flowchart 800 for illustrating example methods executable by external defibrillators according to embodiments.

In an operation at 802, a programmer customizes a particular voice prompt or type of voice prompt to be delivered to a patient by an external defibrillator such as a wearable defibrillator. The programmer may customize the voice prompt(s) by way of a voice prompt customization module in connection with a user interface of the defibrillator, for example. As noted above, the programmer can specify any of a number of characteristics and attributes of each voice prompt or type of voice prompt to be delivered by the defibrillator such as tone quality, language and gender of the spoken voice, and whether the voice prompt gives the appearance of the defibrillator speaking in the first person.

In an operation at 804, the defibrillator stores the customized voice prompts. In certain embodiments, these voice prompts may be stored by a voice prompt database in the defibrillator. The voice prompt database may include a table that specifies the customized attributes for each voice prompt or type of voice prompt.

In an optional operation at 806, a stored voice prompt or type of voice prompt may be altered, regardless of whether the voice prompt was previously customized. The patient may change a certain aspect or attribute of the voice prompt. For example, if the patient has decided that he or she is now more comfortable speaking Spanish than English, he or she can indicate such preference to the voice prompt customization module by way of the user interface so that each subsequent voice prompt delivered to the patient by the defibrillator is in Spanish. In another example where the patient has decided that he or she would rather have the voice prompts be delivered in a female voice rather than a male voice, he or she may indicate such preference to the defibrillator and the preference can then be applied to future voice prompts delivered by the defibrillator. Alternatively, the patient may instruct that the voice prompt or type of voice prompt be deleted.

In an operation at 808, the defibrillator delivers a stored voice prompt, e.g., to the patient, a family member of the patient, or an unidentified rescuer. In certain embodiments, whenever the defibrillator determines that a certain voice prompt is to be delivered, the defibrillator may first consult the voice prompt database to determine which characteristics and attributes are to be applied to the voice prompt during delivery thereof. As noted above, the characteristics and attributes to be applied to the voice prompt may depend on whether the intended recipient is the patient or someone other than a patient, such as a spouse or unidentified rescuer.

In certain embodiments, the patient or other user may press a button, issue a voice command, or provide some other indication to the defibrillator to cause the defibrillator to repeat an issued voice prompt. In such embodiments, the patient or other user may cause the defibrillator to repeat the voice prompt in a different language. Alternatively or in addition thereto, the patient or other user may cause the defibrillator to repeat the voice prompt in a different volume, e.g., louder or softer.

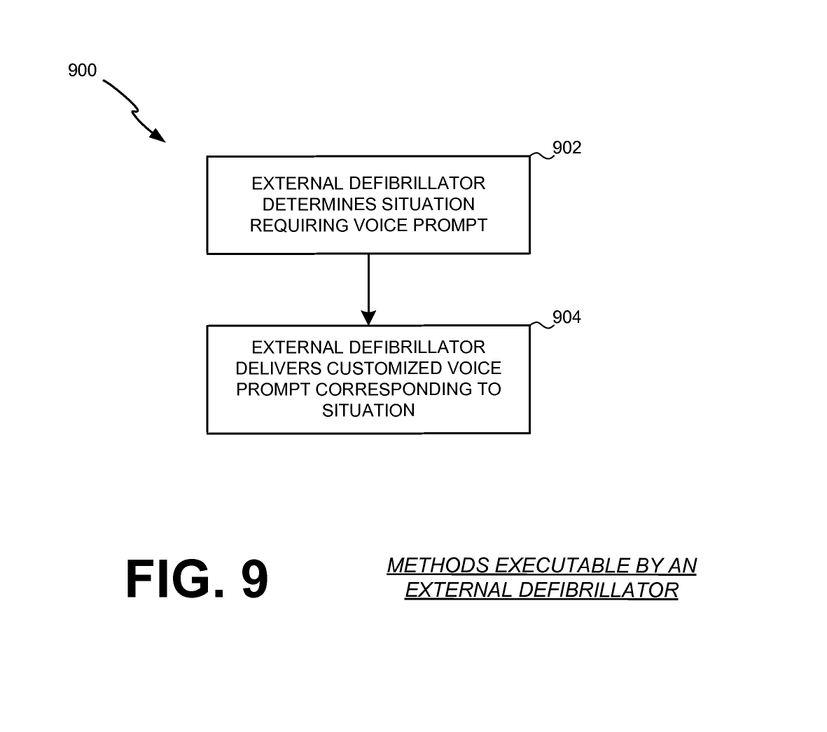

FIG. 9 is a flowchart 900 for illustrating example methods executable by external defibrillators according to embodiments. In an operation at 902, an external defibrillator such as a wearable defibrillator identifies a situation in which the defibrillator is to deliver a voice prompt. For example, the defibrillator may determine the presence of a potential arrhythmia or apparent lack of a heartbeat in the patient. In an operation at 904, the external defibrillator delivers a customized voice prompt that corresponds to the determined event. Specific examples of these operations are now described.

In situations where a shockable arrhythmia in the patient is detected by the external defibrillator, for example, customized voice prompting by the defibrillator can be triggered, e.g., to avoid shocking a conscious person. In other situations, e.g., where the patient is motionless, the defibrillator may deliver a customized voice prompt to determine whether the patient is OK (e.g., “Mr Smith, please press the red button if you are okay.”).

In situations where the defibrillator is to call for help, the defibrillator can be configured to refer to the patient by name (e.g., “Mr. Smith needs help!”). In situations where a rescuer at the scene wants to hear information about the patient, the defibrillator can be configured to provide the patient's medical history responsive to an action by the rescuer, e.g., by pressing a button on the defibrillator.

In situations where the defibrillator is to call an emergency center, e.g., place a 911 call, the device can be configured to provide patient-specific information, such as name, condition, and pertinent medical history, to the emergency center, e.g., 911 operator. For example, the defibrillator may announce that “Mr Smith is experiencing cardiac arrest and has a history of left ventricular cardiomyopathy.” The information provided by the defibrillator to the emergency center can also include a listing of medications currently taken by the patient.

In certain embodiments, the defibrillator can provide patient-specific information on a screen of the device or other display mechanism. Such patient-specific information can be particularly useful for rescuers. For example, the defibrillator can provide the patient's age, weight, known health conditions, number of previous shocks delivered thereto, initial rhythm in the current cardiac arrest, and current medications. This information is often useful to rescuers in determining the proper treatment for the patient. Such information can also be wirelessly transmitted to a 911 operator, sent to an electronic patient care report, sent to another defibrillator or monitor, or sent to another display or data recording device.

In this description, numerous details have been set forth in order to provide a thorough understanding. In other instances, well-known features have not been described in detail in order to not obscure unnecessarily the description.

A person skilled in the art will be able to practice embodiments of the disclosed technology in view of the present description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, certain embodiments may be practiced in combination with other systems.

Other embodiments may include combinations and sub-combinations of features described herein including for example, embodiments that are equivalent to providing or applying a feature in a different order than in a described embodiment, extracting an individual feature from one embodiment and inserting such feature into another embodiment, removing one or more features from an embodiment, or both removing a feature from an embodiment and adding a feature extracted from another embodiment, while providing the advantages of such features incorporated in such combinations and sub-combinations.

What is claimed is:

1. An external medical device, comprising:
a housing;
an energy storage module within the housing for storing an electrical charge;
a defibrillation port within the housing for guiding via electrodes the stored electrical charge to a patient;
a processor within the housing configured to control a voice prompt customization module, in which the voice prompt customization module allows a programmer to customize one or more voice prompts; and
a user interface configured to deliver at least one voice prompt customized by the programmer

2. The external medical device of claim 1, further comprising:
a voice prompt database configured to store the customized voice prompts.

3. The external medical device of claim 2, in which
the user interface delivers the at least one customized voice prompt based on an access to the voice prompt database by the processor.

4. The external medical device of claim 2, in which
the voice prompt database is disposed within the housing.

5. The external medical device of claim 1, in which
the external medical device is configured to be worn by the patient.

6. The external medical device of claim 1, further comprising:
a communication module within the housing configured to establish a wireless connection to another device or system.

7. The external medical device of claim 6, in which
the communication module is configured to make a call to an emergency center.

8. The external medical device of claim 7, in which
the communication module is configured to send information about the patient to the emergency center.

9. The external medical device of claim 1, in which
the voice prompt customization module allows the programmer to specify a wording of at least one voice prompt.

10. The external medical device of claim 9, in which
the specified wording includes the at least one voice prompt being in first person.

11. The external medical device of claim 9, in which
the specified wording includes the at least one voice prompt having a level of verbosity greater than a default verbosity or a level of terseness greater than a default terseness.

12. The external medical device of claim 1, in which
the voice prompt customization module allows the programmer to specify that the at least one voice prompt refer to the patient by his or her name.

13. The external medical device of claim 1, further comprising:
a voice recognition module configured to identify another person by his or her voice.

14. The external medical device of claim 13, in which
the at least one customized voice prompt delivered by the user interface addresses the other person by his or her name.

15. The external medical device of claim 13, in which
the voice prompt customization module allows the programmer to specify a language in which at least one voice prompt is to be delivered based on the identity of the other person.

16. The external medical device of claim 1, in which
the at least one customized voice prompt delivered by the user interface requests a response by the patient.

17. The external medical device of claim 16, in which
the user interface is further configured to receive the requested response from the patient.

18. The external medical device of claim 16, in which
the processor is further configured to cause the user interface to deliver a voice prompt intended for someone other than the patient responsive to the user interface failing to receive the requested response from the patient.

19. The external medical device of claim 1, in which the voice prompt customization module is disposed in the housing.

20. The external medical device of claim 1, in which the programmer is the patient.

21. A method, comprising:
responsive to receiving an instruction from a programmer, a voice prompt customization module of an external defibrillator for a patient customizing a voice prompt;
a voice prompt database of the external defibrillator storing the customized voice prompt; and
a user interface of the external defibrillator delivering the customized voice prompt.

22. The method of claim 21, further comprising:
a voice recognition module of the external defibrillator determining an identity of the person.

23. The method of claim 22, in which
the customized voice prompt delivered to the person by the external defibrillator is based on the determined identity.

24. The method of claim 22, in which
the external defibrillator delivers the customized voice prompt in a language based on the determined identity belonging to someone other than the patient.

25. The method of claim 21, in which
customizing the voice prompt includes specifying that the voice prompt refer to the patient by his or her name.

26. The method of claim 21, in which
customizing the voice prompt includes specifying a wording of the voice prompt.

27. The method of claim 26, in which
specifying the wording of the voice prompt includes specifying that the voice prompt be delivered in first person.

28. The method of claim 26, in which
specifying the wording of the voice prompt includes specifying that the voice prompt have a level of verbosity greater than a default verbosity or a level of terseness greater than a default terseness.

29. The method of claim 21, in which
the customized voice prompt delivered by the user interface requests a response by the patient.

30. The method of claim 29, further comprising:
the user interface delivering a voice prompt intended for someone other than the patient responsive to the user interface failing to receive the requested response from the patient.

31. The method of claim 21, further comprising:
responsive to receiving an instruction from the patient, the voice prompt customization module editing a previously customized voice prompt.

32. The method of claim 21, further comprising:
responsive to receiving an instruction from the patient, the voice prompt customization module deleting a previously customized voice prompt from the voice prompt database.
